(19) United States
(12) Patent Application Publication
(10) Pub. No.: US 2010/0056831 A1
(43) Pub. Date: Mar. 4, 2010

(54) ENERGY-EFFECTIVE PROCESS FOR CO-PRODUCING ETHYLENE AND DIMETHYL ETHER

(75) Inventors: Juntao Liu, Shanghai (CN); Siqing Zhong, Shanghai (CN); Lei Li, Shanghai (CN); Xiaoqi Xin, Shanghai (CN)

Correspondence Address: SCULLY SCOTT MURPHY & PRESSER, PC, 400 GARDEN CITY PLAZA, SUITE 300, GARDEN CITY, NY, 11530, US

(73) Assignees: CHINA PETROLEUM & CHEMICAL CORPORATION, Beijing (CN); SHANGHAI RESEARCH INSTITUTE OF PETROCHEMICAL TECHN, Shanghai (CN)

(21) Appl. No.: 12/377,162

(22) PCT Filed: Aug. 10, 2007
(86) PCT No.: PCT/CN2007/002400

§ 371 Date: Mar. 9, 2009

(30) Foreign Application Priority Data
Aug. 11, 2006 (CN) 200610029971.4
Nov. 2, 2006 (CN) 200610117864.7

(51) Int. Cl. C07C 41/09

(52) U.S. Cl. 568/671

(57) ABSTRACT

The present invention discloses a process for the co-production of ethylene and dimethyl ether, comprising (i) providing a feedstock comprising ethanol and methanol, with a weight ratio of methanol to ethanol being in a range of from 1:10 to 10:1; (ii) feeding the feedstock into a reaction zone containing a solid catalyst to give an effluent, wherein a reaction temperature is in a range of from 200 to 480° C., a reaction pressure is in a range of from 0 to 2 MPa (gauge), and a weight hourly space velocity of the feedstock is in a range of from 0.1 to 10 h⁻¹, and wherein the solid catalyst is selected from the group consisting of alumina catalysts and crystalline aluminosilicate catalysts; and (iii) isolating ethylene and dimethyl ether from the effluent from step (ii).

### CROSS REFERENCE OF RELATED APPLICATIONS

The present application claims the benefit of the Chinese Patent Applications No. 200610029971.4, filed on Aug. 11, 2006; and 200610117864.7, filed on Nov. 2, 2006, which are incorporated herein by reference in their entirety and for all purposes.

### FIELD OF THE INVENTION

The present invention relates to an energy-effective process for the co-production of ethylene and dimethyl ether.

### BACKGROUND OF THE INVENTION

Ethylene is a very important basic organic chemical raw material. In recent years, as the demand to derivatives of ethylene such as polyethylene quickly increases, the demand to ethylene also increases year by year. At present, ethylene is mainly prepared by steam cracking processes of light petroleum cuts, but other processes for preparing ethylene are paid more and more regard as the prices of petroleum and light petroleum cuts have been rising.

A promising approach is to prepare ethylene by dehydrating ethanol. In particular, with the quick development of biotechnology, techniques for producing ethanol through biological processes go ahead ceaselessly so that the source of ethanol is ceaselessly extended and the cost of ethanol becomes more acceptable. Many researches on the preparation of ethylene by dehydrating ethanol have been disclosed in literatures. For example, Zhongqing Zhou, Speciality Petrochemicals, No. 1, 35-37, 1993 reports a research on the preparation of ethylene by dehydrating a feed comprising lower concentration ethanol over a 4 Å molecular sieve catalyst. The results show that, when reaction temperature is in a range of from 250 to 280° C., WHSV of the liquid feed is in a range of from 0.5 to 0.8 h⁻¹, and mass concentration of ethanol in the feed is about 10%, conversion of ethanol may be as high as 99% and selectivity to ethylene may reach 97 to 99%.

Yunxia Yu, Journal of Chemical Industry & Engineering, Vol. 16, No. 2, 8-10, 1995 reports the preparation of NC1301 Catalyst used for preparing ethylene by dehydrating ethanol. Main active component of this catalyst is γ-Al₂O₃. By using this catalyst and under the following conditions: reaction temperature=350 to 440° C., reaction pressure≦0.3 MPaa, and WHSV of ethanol as feedstock=0.3 to 0.6 h⁻¹, a reaction effluent may contain 97.5 to 98.8% of ethylene.

U.S. Pat. No. 4,234,752 discloses a process for preparing ethylene by dehydrating ethanol. Said process utilizes an oxide catalyst and achieves a higher conversion of ethanol at a reaction temperature of from 320 to 450° C. and at a WHSV of from 0.4 to 0.6h⁻¹.

U.S. Pat. No. 4,396,789 discloses a process for preparing ethylene by dehydrating ethanol over an oxide catalyst, wherein a temperature at a reactor inlet is 470° C., and a temperature at a reactor outlet is 360° C.

Chinese Patent Application CN 86101615A discloses a catalyst useful in the preparation of ethylene by dehydrating ethanol. The catalyst comprises ZSM-5 molecular sieve, and gives a higher conversion of ethanol and a higher ethylene yield at a reaction temperature of from 250° C. to 390° C., but exhibits a shorter service lifetime.

Since the reaction of dehydrating ethanol to ethylene is a strongly endothermal reaction, the processes of the prior art for preparing ethylene by dehydrating ethanol suffer generally from lower space velocity of feedstock, higher energy consumption, difficulties associated with reactor enlargement, etc.

Dimethyl ether is a jumped-up basic chemical raw material and finds specific uses in applications such as pharmacy, fuel, pesticide, and the like. Dimethyl ether has a great application prospect as a clean fuel. Furthermore, dimethyl ether may be converted to light olefins through oxygenate-to-olefin processes.

Dimethyl ether is typically produced by the reaction of dehydrating methanol. This reaction is an exothermal reaction so that a large amount of heat has to be removed during the reaction.

### SUMMARY OF THE INVENTION

The inventors have found that the reaction of dehydrating ethanol to prepare ethylene and the reaction of dehydrating methanol to prepare dimethyl ether may be well coupled, and thus an energy-effective process for the co-production of ethylene and dimethyl ether may be provided. This process has the following advantages: reaction temperature is lower, energy consumption is lower, the enlargement of reactor is easy, and operation is simple.

Thus, an object of the invention is to provide a process for the co-production of ethylene and dimethyl ether, comprising the steps of

(i) providing a feedstock comprising ethanol and methanol, with a weight ratio of methanol to ethanol being in a range of from 1:10 to 10:1;

(ii) feeding the feedstock into a reaction zone containing a solid catalyst to give an effluent, wherein a reaction temperature is in a range of from 200 to 480° C., a reaction pressure is in a range of from 0 to 2 MPa (gauge), and a weight hourly space velocity of the feedstock is in a range of from 0.1 to 10 h⁻¹, and wherein the solid catalyst is selected from the group consisting of alumina catalysts and crystalline aluminosilicate catalysts, and

(iii) isolating ethylene and dimethyl ether from the effluent from step (ii).

### DETAILED DESCRIPTION OF THE PREFERRED EMBODIMENTS

In the process according to the invention, ethanol is dehydrated under the action of the solid catalyst to form ethylene:

CH₃CH₂OH→CH₂=CH₂+H₂O,

and methanol is dehydrated under the action of the solid catalyst to form dimethyl ether:

2CH₃OH→(CH₃)₂O+H₂O.

It is well known that the dehydration of ethanol is a strongly endothermal reaction. In the case where pure ethanol is fed, a temperature drop in an adiabatic reactor is about 400° C. Therefore, in fixed bed processes for preparing ethylene by dehydrating ethanol, a shell and tube fixed bed reactor or a multistage fixed bed reactor is generally employed. If a shell and tube fixed bed reactor is employed, for a large scale process for preparing ethylene by dehydrating ethanol, there are problems relating to engineering enlargement and equipment manufacture. Although a multistage fixed bed reactor may maintain the used catalyst in a suitable operation temperature range by supplying heat at a position or positions between the stages, the presence of a larger temperature gradient in the catalyst bed results in that the catalyst cannot function best and that the perfect selectivity to ethylene is hardly achieved. Furthermore, the two kinds of reactor suffer from a common problem that energy consumption is high.

The inventors have noted that the reaction of dehydrating methanol to dimethyl ether is a strongly exothermal reaction and is substantially the same as the reaction of dehydrating ethanol to ethylene in reaction condition, the catalyst used, and the sequent isolation system. Thus, the present invention couples the dehydration reaction of methanol and the dehydration reaction of ethanol, and thereby provides a process for the co-production of ethylene and dimethyl ether. Because the two reactions are coupled in situ in heat, there does not need further supplying or removing a large amount of heat. Hence, the process is energy-effective, resulting in that the process flow is simplified, equipment investment is reduced, and the reactor can be easily enlarged.

There is not a specific limitation to the source of ethanol and methanol used as feedstock in the process of the invention. From the view point of matching reaction heat, the weight ratio of methanol to ethanol in the feedstock may be in a range of from 1:10 to 10:1, preferably from 1:5 to 8:1, more preferably from 1:2 to 6:1, and most preferably from 1:1 to 5:1.

The catalyst useful in the process of the invention may be selected from the group consisting of alumina catalysts and crystalline aluminosilicate catalysts, which are known by those skilled in the art. The alumina catalysts comprise preferably γ-Al₂O₃. Crystalline aluminosilicate catalysts comprise preferably at least one selected from the group consisting of ZSM molecular sieves, β-zeolites and mordenite. In a preferred embodiment, the solid catalyst comprises a ZSM molecular sieve, especially a ZSM-5 molecular sieve, having a molar ratio of SiO₂ to Al₂O₃ of from 20 to 500, and preferably from 30 to 200. In addition to the alumina or the crystalline aluminosilicate, the catalyst may further comprise a conventional binder.

The process according to the invention may be carried out under the following reaction conditions: a reaction temperature in a range of from 200 to 480° C., a reaction pressure in a range of from 0 to 2 MPa (gauge), and a WHSV of the feedstock in a range of from 0.1 to 10 h⁻¹. The reaction conditions may be further optimized according to the selected catalyst. When the solid catalyst is an alumina catalyst, the reaction temperature is preferably in a range of from 300 to 480° C., and more preferably from 350 to 430° C.; the WHSV of the feedstock is preferably in a range of from 0.5 to 5 h⁻¹; and the reaction pressure is preferably in a range of from 0.1 to 1 MPa (gauge). When the solid catalyst is a crystalline aluminosilicate catalyst, the reaction temperature is preferably in a range of from 200 to 400° C., and more preferably from 230 to 350° C.; the WHSV of the feedstock is preferably in a range of from 0.5 to 5 h⁻¹; and the reaction pressure is preferably in a range of from 0.01 to 1.0 MPa (gauge).

In an embodiment, at least a part of the obtained dimethyl ether is further converted to olefins, especially light olefins, mainly ethylene and propylene, through an oxygenate-to-olefin process. The oxygenate-to-olefin processes are well known by those skilled in the art. See, for example, CN96115333.4, CN00802040.X, CN01144188.7, CN 200410024734.X, and CN92109905.3.

The process of the invention allows the reaction to proceed at a lower temperature, for example about 250° C., under a higher space velocity of the feedstock, for example more than 5 h⁻¹. The reduction of the reaction temperature may markedly lower energy consumption in the operation, aid to reduce side reactions, and lower the rate of catalyst coking to thereby effectively prolong the service lifetime of the catalyst. The enhancement of the space velocity of the feedstock may enhance throughput per unit volume of the reactor. Furthermore, the heat released by the reaction of dehydrating methanol compensates the heat taken up by the reaction of dehydrating ethanol so that a non-shell and tube type monostage adiabatic fixed bed reactor may be employed to carry out the reaction of dehydrating ethanol to ethylene. As a result, the difficulty relating to reactor enlargement is greatly reduced and energy consumption in the operation is further reduced.

By the process according to the invention, higher conversion of ethanol, for example approximately 100%, higher selectivity to ethylene, for example more than 96%, and higher selectivity to dimethyl ether, for example more than 90%, are achieved.

### EXAMPLES

The following examples are given for further illustrating the invention, but do not make limitation to the invention in any way.

### Example 1

Ten grams of γ-Al₂O₃ catalyst having a specific surface area of 200 m²/g and an alumina content of 99.7 wt. % were charged into a fixed bed reactor having an inner diameter of 22 mm, and then activated in a nitrogen flow at 550° C. for 2 h. After allowing the temperature inside the reactor to lower to reaction temperature, a feedstock consisting of methanol and ethanol (mass ratio of methanol to ethanol=2:1) was continuously fed into the reactor and allowed to react under the following conditions: reaction temperature=360° C., WHSV of the feedstock=1.5 h⁻¹, and reaction pressure=0.02 MPa (gauge). Effluent of the reactor was analyzed, and it was found that conversion of ethanol was approximatively 100%, selectivity to ethylene was 99.6%, conversion of methanol was 78.1%, and selectivity to dimethyl ether was 98.1%.

### Examples 2 to 12

Experiments were carried out following the procedure as described in the Example 1 under the conditions as set forth in the Table 1 below. The results are shown in the Table 1.

TABLE 1

| | | | | | Results | | | |
|---|---|---|---|---|---|---|---|---|
| | Weight Ratio | Reactor | WHSV of | Reaction | Conversion | Selectivity | Conversion | Selectivity |
| | of Methanol | Temperature | Feedstock | Pressure | of Ethanol | to Ethylene | of Methanol | to Dimethyl Ether |
| Ex. No. | to Ethanol | ° C. | h⁻¹ | MPa(gauge) | % | % | % | % |
| 1 | 2 | 360 | 1.5 | 0.02 | 100 | 99.6 | 78.1 | 98.1 |
| 2 | 1 | 380 | 0.8 | 0.2 | 100 | 96.8 | 74.3 | 92.0 |
| 3 | 2 | 330 | 4.0 | 0.05 | 80.7 | 90.2 | 80.8 | 95.4 |
| 4 | 1/6 | 400 | 3.5 | 0.2 | 99.8 | 94.3 | 53.4 | 94.7 |
| 5 | 6 | 450 | 0.5 | 1.5 | 100 | 88.4 | 77.2 | 91.6 |
| 6 | 1/3 | 370 | 10.0 | 0.03 | 92.4 | 93.9 | 60.9 | 94.1 |
| 7 | 1/2 | 360 | 3.0 | 1.7 | 100 | 89.2 | 69.9 | 94.6 |
| 8 | 4 | 380 | 2.0 | 0.3 | 99.5 | 92.4 | 72.1 | 91.0 |
| 9 | 6 | 360 | 2.0 | 0.4 | 98.7 | 93.8 | 76.7 | 92.3 |
| 10 | 8 | 400 | 6.0 | 0.2 | 100 | 88.7 | 52.5 | 90.7 |
| 11 | 10 | 400 | 4.0 | 0.15 | 100 | 89.6 | 54.8 | 91.9 |
| 12 | 1/2 | 480 | 8.0 | 0.5 | 100 | 86.1 | 62.1 | 88.3 |

### Example 13

Ten grams of γ-Al₂O₃ catalyst having a specific surface area of 200 m²/g and an alumina content of 99.7 wt. % were charged into an adiabatic fixed bed reactor having an inner diameter of 22 mm, and then activated in a nitrogen flow at 550° C. for 2 h. After allowing the temperature inside the reactor to lower to reaction temperature, a feedstock consisting of methanol and ethanol (mass ratio of methanol to ethanol=1:2) was continuously fed into the reactor and allowed to react under the following conditions: temperature at reactor inlet=400° C., WHSV of the feedstock=3.6 h⁻¹, and reaction pressure=0.2 MPa (gauge). The temperature at reactor outlet was 328° C. Effluent of the reactor was analyzed, and it was found that conversion of ethanol was approximatively 100%, selectivity to ethylene was 95.7%, conversion of methanol was 80.7%, and selectivity to dimethyl ether was 93.2%.

### Example 14

10 g of γ-Al₂O₃ catalyst having a specific surface area of 200 m²/g and an alumina content of 99.7 wt. % was charged into an adiabatic fixed bed reactor having an inner diameter of 22 mm, and then activated in a nitrogen flow at 550° C. for 2 h. After allowing the temperature inside the reactor to lower to reaction temperature, a feedstock consisting of methanol and ethanol (mass ratio of methanol to ethanol=4:1) was continuously fed into the reactor and allowed to react under the following conditions: temperature at reactor inlet=360° C., WHSV of the feedstock=4 h⁻¹, and reaction pressure=0.06 MPa (gauge). The temperature at reactor outlet was 362° C. Effluent of the reactor was analyzed, and it was found that conversion of ethanol was approximatively 100%, selectivity to ethylene was 97.7%, conversion of methanol was 81.2%, and selectivity to dimethyl ether was approximatively 100%.

### Example 15

100 g of ZSM-5 molecular sieve having a SiO₂/Al₂O₃ molar ratio of 40 was mixed with 60 g of a silica sol (having a silica content of 30 wt. %), and then the mixture was extruded. The extrudates were dried at 180° C. for 6 h, and then calcined at 500° C. for 4 h, to give a ZSM-5 molecular sieve catalyst.

3 g of the prepared ZSM-5 molecular sieve catalyst was charged into a fixed bed reactor having an inner diameter of 18 mm, and then activated in a nitrogen flow at 550° C. for 2 h. After allowing the temperature inside the reactor to lower to reaction temperature, a feedstock consisting of methanol and ethanol (mass ratio of methanol to ethanol=2:1) was continuously fed into the reactor and allowed to react under the following conditions: reaction temperature=250° C., WHSV of the feedstock=3 h⁻¹, and reaction pressure=0.02 MPa (gauge). Effluent of the reactor was analyzed, and it was found that conversion of ethanol was 99.2%, selectivity to ethylene was 95.4%, conversion of methanol was 78.1%, and selectivity to dimethyl ether was 90.4%.

### Examples 16 to 26

Experiments were carried out following the procedure as described in the Example 15 under the conditions as set forth in the Table 2 below. The results are shown in the Table 2.

TABLE 2

| | | | | | | Results | | | |
|---|---|---|---|---|---|---|---|---|---|
| | | Weight Ratio | Reactor | WHSV of | Reaction | Conversion | Selectivity | Conversion | Selectivity |
| | | of Methanol | Temperature | Feedstock | Pressure | of Ethanol | to Ethylene | of Methanol | to Dimethyl Ether |
| Ex. | Catalyst | to Ethanol | ° C. | h⁻¹ | MPa(gauge) | % | % | % | % |
| 16 | ZSM-5 (molar ratio of | 1 | 280 | 1.0 | 0.2 | 99.8 | 96.2 | 80.1 | 91.3 |
| | SiO₂/Al₂O₃ = 40) | | | | | | | | |
| 17 | ZSM-5 (molar ratio of | 1/2 | 300 | 2.2 | 0.05 | 99.7 | 96.7 | 82.4 | 90.9 |
| | SiO₂/Al₂O₃ = 60) | | | | | | | | |
| 18 | ZSM-5 (molar ratio of | 6 | 380 | 10 | 0.8 | 94.1 | 79.1 | 81.4 | 83.6 |
| | SiO₂/Al₂O₃ = 180) | | | | | | | | |
| 19 | ZSM-5 (molar ratio of | 1/10 | 260 | 4.5 | 0.5 | 98.7 | 90.6 | 60.4 | 87.7 |
| | SiO₂/Al₂O₃ = 120) | | | | | | | | |
| 20 | ZSM-5 (molar ratio of | 1/3 | 320 | 10 | 0.03 | 96.8 | 87.8 | 68.4 | 89.1 |
| | SiO₂/Al₂O₃ = 20) | | | | | | | | |
| 21 | ZSM-5 (molar ratio of | 1/2 | 260 | 3.5 | 0.7 | 100 | 95.6 | 76.0 | 90.2 |
| | SiO₂/Al₂O₃ = 52) | | | | | | | | |
| 22 | β-zeolite (molar ratio of | 1/2 | 280 | 1.5 | 2.0 | 99.5 | 80.5 | 76.3 | 89.3 |
| | SiO₂/Al₂O₃ = 300) | | | | | | | | |
| 23 | Mordenite (molar ratio of | 3 | 352 | 0.5 | 1.0 | 100 | 87.0 | 81.1 | 90.2 |
| | SiO₂/Al₂O₃ = 450) | | | | | | | | |
| 24 | ZSM-48 (molar ratio of | 5 | 300 | 9 | 0 | 100 | 87.4 | 84.7 | 91.5 |
| | SiO₂/Al₂O₃ = 150) | | | | | | | | |
| 25 | ZSM-11 (molar ratio of | 1.25 | 290 | 2.25 | 0.5 | 98.2 | 88.3 | 81.3 | 87.7 |
| | SiO₂/Al₂O₃ = 80) | | | | | | | | |
| 26 | SAPO-34 | 2 | 300 | 2.4 | 0.1 | 99.5 | 90.6 | 82.4 | 81.4 |

### Example 27

100 g of ZSM-5 molecular sieve having a SiO₂/Al₂O₃ molar ratio of 50 was mixed with 60 g of a silica sol (having a silica content of 30 wt. %), and then the mixture was extruded. The extrudates were dried at 180° C. for 6 h, and then calcined at 500° C. for 4 h, to give a ZSM-5 molecular sieve catalyst.

3 g of the prepared ZSM-5 molecular sieve catalyst was charged into an adiabatic fixed bed reactor having an inner diameter of 18 mm, and then activated in a nitrogen flow at 550° C. for 2 h. After allowing the temperature inside the reactor to lower to reaction temperature, a feedstock consisting of methanol and ethanol (mass ratio of methanol to ethanol=2:1) was continuously fed into the reactor and allowed to react under the following conditions: temperature at reactor inlet=360° C., WHSV of the feedstock=3 h⁻¹, and reaction pressure=0.2 MPa (gauge). The temperature at reactor outlet was 280° C. Effluent of the reactor was analyzed, and it was found that conversion of ethanol was approximatively 100%, selectivity to ethylene was 91.3%, conversion of methanol was 83.7%, and selectivity to dimethyl ether was 90.8%.

### Example 28

100 g of ZSM-5 molecular sieve having a SiO₂/Al₂O₃ molar ratio of 80 was mixed with 60 g of a silica sol (having a silica content of 30 wt. %), and then the mixture was extruded. The extrudates were dried at 180° C. for 6 h, and then calcined at 500° C. for 4 h, to give a ZSM-5 molecular sieve catalyst.

3 g of the prepared ZSM-5 molecular sieve catalyst was charged into an adiabatic fixed bed reactor having an inner diameter of 18 mm, and then activated in a nitrogen flow at 550° C. for 2h. After allowing the temperature inside the reactor to lower to reaction temperature, a feedstock consisting of methanol and ethanol (mass ratio of methanol to ethanol=4:1) was continuously fed into the reactor and allowed to react under the following conditions: temperature at reactor inlet=300° C., WHSV of the feedstock=0.8 h⁻¹, and reaction pressure=0.06 MPa (gauge). The temperature at reactor outlet was 300° C. Effluent of the reactor was analyzed, and it was found that conversion of ethanol was approximatively 100%, selectivity to ethylene was 92.3%, conversion of methanol was 84.2%, and selectivity to dimethyl ether was 91.3%.

The patents, patent applications, non-patent literatures and testing methods cited in the specification are incorporated herein by reference.

While the invention has been described with reference to exemplary embodiments, it will be understood by those skilled in the art that various changes and modifications may be made without departing from the spirit and scope of the invention. Therefore, the invention is not limited to the particular embodiments disclosed as the best mode contemplated for carrying out this invention, but the invention will include all embodiments falling within the scope of the appended claims.

1. A process for the co-production of ethylene and dimethyl ether, comprising the steps of
(i) providing a feedstock comprising ethanol and methanol, with a weight ratio of methanol to ethanol being in a range of from 1:10 to 10:1;
(ii) feeding the feedstock into a reaction zone containing a solid catalyst to give an effluent, wherein a reaction temperature is in a range of from 200 to 480° C., a reaction pressure is in a range of from 0 to 2 MPa (gauge), and a weight hourly space velocity of the feedstock is in a range of from 0.1 to 10 h⁻¹, and wherein the solid catalyst is selected from the group consisting of alumina catalysts and crystalline aluminosilicate catalysts; and
(iii) isolating ethylene and dimethyl ether from the effluent from step (ii).

2. The process according to claim 1, wherein the weight ratio of methanol to ethanol is in a range of from 1:5 to 8:1.

3. The process according to claim 1, wherein the weight ratio of methanol to ethanol is in a range of from 1:2 to 6:1.

4. The process according to claim 1, wherein the solid catalyst is an alumina catalyst, and the reaction temperature is in a range of from 300 to 480° C.

5. The process according to claim 4, wherein the reaction temperature is in a range of from 350 to 430° C., the WHSV of the feedstock is in a range of from 0.5 to 5 h⁻¹, and the reaction pressure is in a range of from 0.1 to 1 MPa (gauge).

6. The process according to claim 1, wherein the solid catalyst is an alumina catalyst comprising γ-Al₂O₃.

7. The process according to claim 1, wherein the solid catalyst is a crystalline aluminosilicate catalyst, and the reaction temperature is in a range of from 200 to 400° C.

8. The process according to claim 7, wherein the reaction temperature is in a range of from 230 to 350° C., the WHSV of the feedstock is in a range of from 0.5 to 5 h⁻¹, and the reaction pressure is in a range of from 0.01 to 1.0 MPa (gauge).

9. The process according to claim 1, wherein the solid catalyst is a crystalline aluminosilicate catalyst comprising at least one selected from the group consisting of ZSM molecular sieves, β-zeolites and mordenite.

10. The process according to claim 9, wherein the solid catalyst is a crystalline aluminosilicate catalyst comprising a ZSM molecular sieve having a molar ratio of SiO₂ to Al₂O₃ of from 20 to 500.

11. The process according to claim 10, wherein the solid catalyst is a crystalline aluminosilicate catalyst comprising a ZSM-5 molecular sieve having a molar ratio of SiO₂ to Al₂O₃ of from 30 to 200.

12. The process according to claim 1, further comprising converting at least a part of the obtained dimethyl ether to light olefins by an oxygenate-to-olefin process.

13. The process according to claim 12, wherein the light olefins are ethylene and/or propylene.
